SEQUENCE LISTING (6 sequences)

SEQ ID NO: 1 - protein, 243 aa, Aspergillus oryzae
  SIGNAL (1)..(22)
  PROPEP (23)..(37)
  CHAIN (38)..(243): mature polypeptide
MQLTKSLLVFALYMFGTQHVLAVPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 2 - protein, 206 aa, Aspergillus oryzae
  CHAIN (1)..(206): mature polypeptide
ALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 3 - protein, 204 aa, Aspergillus oryzae
  CHAIN (1)..(204): mature polypeptide
KTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 4 - protein, 205 aa, Trichoderma harzianum
  SIGNAL (1)..(17)
  CHAIN (18)..(205): mature polypeptide
MKLSISVALTSAIAVLAAPAPMPTPPGIPTESSARTQLAGLTVAVAGSGTGYSRDLFPTWDAISGNCNAREYVLKRDGEGVQVNNACESQSGTWISPYDNASFTNASSLDIDHMVPLKNAWISGASSWTTAQREALANDVSRPQLWAVSASANRSKGDRSPDQWKPPLTSFYCTYAKSWIDVKSFYKLTITSAEKTALSSMLDTC

SEQ ID NO: 5 - protein, 142 aa, Bacillus licheniformis
  SIGNAL (1)..(33)
  CHAIN (34)..(142): mature polypeptide
MIKKWAVHLLFSALVLLGLSGGAAYSPQHAEGAARYDDILYFPASRYPETGAHISDAIKAGHSDVCTIERSGADKRRQESLKGIPTKPGFDRDEWPMAMCEEGGKGASVRYVSSSDNRGAGSWVGNRLSGFADGTRILFIVQ

SEQ ID NO: 6 - protein, 136 aa, Bacillus subtilis
  SIGNAL (1)..(26)
  CHAIN (27)..(136): mature polypeptide
MKKWMAGLFLAAAVLLCLMVPQQIQGASSYDKVLYFPLSRYPETGSHIRDAIAEGHPDICTIDRDGADKRREESLKGIPTKPGYDRDEWPMAVCEEGGAGADVRYVTPSDNRGAGSWVGNQMSSYPDGTRVLFIVQ

(19) United States
(12) Patent Application Publication
(10) Pub. No.: US 2018/0112156 A1
(43) Pub. Date: Apr. 26, 2018

(54) LAUNDRY METHOD, USE OF POLYPEPTIDE AND DETERGENT COMPOSITION

(71) Applicant: NOVOZYMES A/S, Bagsvaerd (DK)

(72) Inventors: Klaus Gori, Dyssegaard (DK); Lilian Eva Tang Baltsen, Bagsvaerd (DK)

(73) Assignee: NOVOZYMES A/S, Bagsvaerd (DK)

(21) Appl. No.: 15/561,853

(22) PCT Filed: Apr. 11, 2016
(86) PCT No.: PCT/EP2016/057899

§ 371 Date: Sep. 26, 2017

(30) Foreign Application Priority Data
Apr. 10, 2015 (EP) 15163228.8

(51) Int. Cl. C11D 3/386; C11D 3/39; C11D 3/42; C12N 9/22; C11D 1/02; C11D 11/00

(52) U.S. Cl. CPC C11D 3/38636; C11D 3/3915; C11D 3/3917; C11D 11/0017; C12N 9/22; C11D 1/02; C11D 3/42

(57) ABSTRACT

The present invention concerns a method for laundering a textile, the use of a polypeptide having DNase activity and a detergent composition comprising a polypeptide having deoxyribonuclease (DNase) activity.

### REFERENCE TO A SEQUENCE LISTING

This application contains a Sequence Listing in computer readable form, which is incorporated herein by reference.

### FIELD OF THE INVENTION

The present invention concerns a method for laundering a textile, the use of a polypeptide having DNase activity and a detergent composition comprising a polypeptide having deoxyribonuclease (DNase) activity.

### BACKGROUND OF INVENTION

Bleaching systems are present in certain detergents to bleach particular stains such as red wine, tea, coffee, fruit juices, grass, carrot, or tomato sauce, whether on clothes or tableware. Bleaching systems also help to maintain the whiteness and brightness of garments, in particular whites. Optical brighteners overlay the textile and convert UV to visible light in the blue or yellow spectra and the result is that greyness and discoloration of e.g. textiles is hidden.

However, many bleaching compounds suffer from poor compatibility with other detergent ingredients during storage, in particular with enzymes. One way of getting round this is adding the bleaching compounds after the enzymes have been allowed to work for a time period e.g. As described in international patent application WO 2012/028482.

In order for a bleach compound to actually bleach a textile, it is important the surface is uniformly clean so the bleach compound is exposed to the whole surface of the textile.

Soiled textiles may absorb the optical brighteners differently with different efficiency and thereby also give the textile a mottled appearance.

The present invention solves the problems of the prior art.

### SUMMARY OF THE INVENTION

The present invention concerns a method for laundering a textile comprising the steps of:
a) Contacting the textile to a wash liquor comprising a polypeptide having DNase activity, an anionic surfactant a bleach system comprising tetraacetylethylenediamine (TAED) or 4-(nonanoyloxy)benzene-1-sulfonate (NOBS) and optionally an optical brightener; and
b) Optionally rinsing the textile,
wherein the wash liquor has a temperature in the range of 10-60° C.

The invention further concerns the use of a polypeptide having DNase activity for preparing a textile surface for receiving a bleach system comprising tetraacetylethylenediamine (TAED) or 4-(nonanoyloxy)benzene-1-sulfonate (NOBS). Further is claimed a detergent composition comprising a polypeptide having deoxyribonuclease (DNase) activity, a anionic surfactant, and a bleach system comprising tetraacetylethylenediamine (TAED) or 4-(nonanoyloxy)benzene-1-sulfonate (NOBS).

### Definitions

Bacterial: In the context of the present invention, the term “bacterial” in relation to polypeptide (such as an enzyme, e.g. a DNase) refers to a polypeptide encoded by and thus directly derivable from the genome of a bacteria, where such bacteria has not been genetically modified to encode said polypeptide, e.g. by introducing the encoding sequence in the genome by recombinant DNA technology. In the context of the present invention, the term “bacterial DNase” or “polypeptide having DNase activity obtained from a bacterial source” or “polypeptide is of bacterial origin” thus refers to a DNase encoded by and thus directly derivable from the genome of a bacterial species, where the bacterial species has not been subjected to a genetic modification introducing recombinant DNA encoding said DNase. Thus, the nucleotide sequence encoding the bacterial polypeptide having DNase activity is a sequence naturally in the genetic background of a bacterial species. The bacterial polypeptide having DNase activity encoding by such sequence may also be referred to a wild type DNase (or parent DNase). In a further aspect, the invention provides polypeptides having DNase activity, wherein said polypeptides are substantially homologous to a bacterial DNase. In the context of the present invention, the term “substantially homologous” denotes a polypeptide having DNase activity which is at least 80%, preferably at least 85%, more preferably at least 90%, more preferably at least 95%, even more preferably at least 96%, 97%, 98%, and most preferably at least 99% identical to the amino acid sequence of a selected bacterial DNase.

Biofilm: A biofilm is any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium.

Bacteria living in a biofilm usually have significantly different properties from planktonic bacteria of the same species, as the dense and protected environment of the film allows them to cooperate and interact in various ways. One benefit of this environment is increased resistance to detergents and antibiotics, as the dense extracellular matrix and the outer layer of cells protect the interior of the community.

On laundry biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp.

Color difference (L value): A Lab color space is a color-opponent space with dimension L for lightness. L value, L* represents the darkest black at L*=0, and the brightest white at L*=100. In the context of the present invention L value is also referred to as color difference.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Detergent components: The term “detergent components” is defined herein to mean the types of chemicals which can be used in detergent compositions. Examples of detergent components are alkalis, surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants and solubilizers.

Detergent composition: The term “detergent composition” refers to compositions that find use in the removal of undesired compounds from items to be cleaned, such as textiles. The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pre-treatment). In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidaes, haloperoxygenases, catalases and mannanases, or any mixture thereof), and/or detergent components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

DNase: The term “DNase” means a polypeptide with DNase activity that catalyzes the hydrolytic cleavage of phosphodiester linkages in the DNA backbone, thus degrading DNA. For purposes of the present invention, DNase activity is determined according to the procedure described in the Assay I. In one embodiment of the present invention, the DNase activity of polypeptide having is at least 105%, e.g., at least 110%, at least 120%, at least 130%, at least 140%, at least 160%, at least 170%, at least 180%, or at least 200% with reference to the DNase activity of the mature polypeptide of SEQ ID NO: 1, a polypeptide comprising or consisting of the sequence set forth in SEQ ID NO: 2, a polypeptide comprising or consisting of the sequence set forth in SEQ ID NO: 3, a polypeptide comprising or consisting of the mature polypeptide of SEQ ID NO: 4, a polypeptide comprising or consisting of the mature polypeptide of SEQ ID NO: 5 or a polypeptide comprising or consisting of the mature polypeptide of SEQ ID NO: 6.

Enzyme detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-back staining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides.

Fungal: In the context of the present invention, the term “fungal” in relation to polypeptide (such as an enzyme, e.g. a DNase) refers to a polypeptide encoded by and thus directly derivable from the genome of a fungus, where such fungus has not been genetically modified to encode said polypeptide, e.g. by introducing the encoding sequence in the genome by recombinant DNA technology. In the context of the present invention, the term “fungal DNase” or “polypeptide having DNase activity obtained from a fungal source” or “polypeptide is of fungal origin” thus refers to a DNase encoded by and thus directly derivable from the genome of a fungal species, where the fungal species has not been subjected to a genetic modification introducing recombinant DNA encoding said DNase. Thus, the nucleotide sequence encoding the fungal polypeptide having DNase activity is a sequence naturally in the genetic background of a fungal species. The fungal polypeptide having DNase activity encoding by such sequence may also be referred to a wild type DNase (or parent DNase). In a further aspect, the invention provides polypeptides having DNase activity, wherein said polypeptides are substantially homologous to a fungal DNase. In the context of the present invention, the term “substantially homologous” denotes a polypeptide having DNase activity which is at least 80%, preferably at least 85%, more preferably at least 90%, more preferably at least 95%, even more preferably at least 96%, 97%, 98%, and most preferably at least 99% identical to the amino acid sequence of a selected fungal DNase.

Hard surface: The term “hard surface” is defined herein as hard surfaces including floors, tables, walls, roofs etc. as well as surfaces of hard objects such as cars (car wash) and dishes (dishware). Dishware includes but is not limited to plates, cups, glasses, bowls, cutlery such as spoons, knives, forks, serving utensils, ceramics, plastics, metals, china, glass and acrylics.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Mature polypeptide: The term “mature polypeptide” means a polypeptide in its final form following translation and any post-translational modifications, such as N-terminal processing, C-terminal truncation, glycosylation, phosphorylation, etc. In one embodiment, the mature polypeptide is amino acids 38 to 243 of SEQ ID NO: 1 and amino acids 1 to 22 of SEQ ID NO: 1 are a signal peptide and amino acids 23 to 37 of SEQ ID NO: 1 are a propeptide. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide. In one embodiment, a mature polypeptides contains up to 206 (such as 204) consecutive amino acid residues of the sequence set forth in SEQ ID NO: 1 or SEQ ID NO: 2 (e.g., amino acids 38 to 243 of SEQ ID NO: 1 or amino acids 1 to 206 of SEQ ID NO: 2 or amino acids 1 to 204 of SEQ ID NO: 3), or up to 204 amino acid residues (e.g., amino acids 40 to 243 of SEQ ID NO: 1). In another embodiment, the mature polypeptide consists of the of the amino acid sequence set forth in SEQ ID NO: 2 or SEQ ID NO: 3. In yet another embodiment, the mature polypeptide comprises or consists of the consecutive amino acid residues 18 to 205 of SEQ ID NO: 4. In one embodiment, the mature polypeptide comprises or consists of the consecutive amino acid residues 34 to 142 of SEQ ID NO: 5. In one embodiment, the mature polypeptide comprises or consists of the consecutive amino acid residues 27 to 136 of SEQ ID NO: 6.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well. In the context of the present invention, the term “textile” also covers fabrics.

Variant: The term “variant” means a polypeptide having same activity as the parent enzyme comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. In the context of the present invention, a variant of an identified DNase has the enzymatic activity of the parent, i.e. the capacity of catalyzing the hydrolytic cleavage of phosphodiester linkages in the DNA backbone (deoxyribonuclease activity). In one embodiment, the deoxyribonuclease activity of the variant is increased with reference to the parent DNase, e.g. the mature polypeptide of a polypeptide having deoxyribonuclease activity is selected from the group consisting of a polypeptide comprising or consisting of the mature polypeptide of SEQ ID NO: 1, a polypeptide comprising or consisting of the sequence set forth in SEQ ID NO: 2, a polypeptide comprising or consisting of the sequence set forth in SEQ ID NO: 3, a polypeptide comprising or consisting of the mature polypeptide of SEQ ID NO: 4, a polypeptide comprising or consisting of the mature polypeptide of SEQ ID NO: 5 or a polypeptide comprising or consisting of the mature polypeptide of SEQ ID NO: 6.

Wash cycle: The term “wash cycle” is defined herein as a washing operation wherein textiles are immersed in the wash liquor, mechanical action of some kind is applied to the textile in order to release stains and to facilitate flow of wash liquor in and out of the textile and finally the superfluous wash liquor is removed. After one or more wash cycles, the textile is generally rinsed and dried.

Wash liquor: The term “wash liquor” is intended to mean the solution or mixture of water and detergents optionally including enzymes used for laundering textiles.

### DETAILED DESCRIPTION OF THE INVENTION

The inventors have surprisingly found that it is possible to wash with enzymes having DNase activity at the same time as washing with bleach system comprising tetraacetylethylenediamine (TAED) or 4-(nonanoyloxy)benzene-1-sulfonate (NOBS) without the polypeptides being negatively affected by the bleaching system. Some aspects of the invention relates to a detergent composition comprising a DNase and a bleach system, e.g. comprising tetraacetylethylenediamine (TAED) or 4-(nonanoyloxy)benzene-1-sulfonate (NOBS).

Some aspects of the invention relate to detergent compositions comprising an optical brightener (fluorescent whitening agents). Optical brighteners, optical brightening agents (OBAs), fluorescent brightening agents (FBAs) or fluorescent whitening agents (FWAs) absorb light in the UV and violet region and re-emit light in the blue region. The term “optical brighteners” has its common meaning in the field of laundry detergents and includes optical brighteners, optical brightening agents (OBAs), fluorescent brightening agents (FBAs) and fluorescent whitening agents (FWAs). Optical brighteners reflect light in a way that makes fabric look white e.g. by reducing the yellowish appearance of textiles. Textiles washed with detergents containing optical brighteners deposit reflective particles on the textiles which make them seem whiter. A problem with optical brighteners is that they prefer an even surface for optimum performance. Textiles laundered with a DNase polypeptide of the invention has the advantage of having a more even the surface compared to textiles not laundered with DNase. The combination of DNase and optical brighteners are therefore particularly advantageous as the DNase has a positive effect on the performance of the optical brighteners. In some aspects of the invention the DNases have a synergy with the optical brightener e.g. the effect of an optical brightener in a composition comprising at least one DNase(s) is synergistic compared to the effect of an optical brightener in a composition not comprising DNase(s). One advantage of using a DNase in combination with an optical brightener is that the effect e.g. the synergistic effect, of DNases in combination with optical brighteners for treating surfaces such as textiles allows the consumer to use less optical brightener and at the same time keeping the textiles white simply by increasing the effect of the optical brightener. Thus, some aspects of the invention relates to the use of a DNase for preparing a surface for application of an optical brightener, wherein the surface is a textile. Some aspects of the invention relates to a method of laundering comprising the steps of:
a) Contacting the textile with a detergent composition comprising a polypeptide having DNase activity, optionally a anionic surfactant, optionally a bleach system, e.g. tetraacetylethylenediamine (TAED) or 4-(nonanoyloxy)benzene-1-sulfonate (NOBS) and an optical brightener; and
b) Optionally rinsing the textile.

Some aspects of the invention further relate to detergent compositions comprising a polypeptide having DNase activity and at least one optical brightener, optionally the detergent may comprise a surfactant such as an anionic surfactant and/or optionally a bleach system e.g. tetraacetylethylenediamine (TAED) or 4-(nonanoyloxy)benzene-1-sulfonate (NOBS).

Preferably, the concentration of optical brightener in the detergent is at a level of about 0.01% to about 0.5%. Any optical brightener and/or fluorescent whitening agent suitable for use in the detergent composition of the invention may be used in the composition of the present invention. Some preferred fluorescent whitening agents are those belonging to the classes of diaminostilbene-sulfonic acid derivatives, diarylpyrazoline derivatives and bisphenyl-distyryl derivatives. Some preferred diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01 wt % of the total wt % of the detergent, from 0.05 wt %, from about 0.1 wt % or even from about 0.2 wt % to upper levels of 0.5 or even 0.75 wt %.

The inventors have found that by laundering a textile in a method comprising the steps of:
a) Contacting the textile to a wash liquor comprising a polypeptide having DNase activity, an anionic surfactant and a bleach system comprising tetraacetylethylenediamine (TAED) or 4-(nonanoyloxy)benzene-1-sulfonate (NOBS); and
b) Optionally rinsing the textile, wherein the wash liquor has a temperature in the range of 10-60° C.

The textile becomes cleaner than when washed without the combination of the polypeptide, the surfactant and the bleach system.

The textile can be rinsed with water or with water comprising a conditioner.

The temperature of the wash liquor during the laundering process is important. The inventors have found that the bleaching effect is found when the temperature is below 60° C. The inventors have found that when washing a textile according to the inventive method with the combination of a polypeptide having DNase activity, an anionic surfactant and a bleach system then the washed textile is cleaner than washed with conventional laundering methods. Thus, the inventors have found that the activity of polypeptides having DNase activity is not affected by the presence of a bleach system comprising tetraacetylethylenediamine (TAED) or 4-(nonanoyloxy)benzene-1-sulfonate (NOBS), even at high concentrations of the bleach system.

In one embodiment of the invention, the temperature of the wash liquor is in the range of 10-50° C., in the range of 10-45° C., in the range of 10-40° C., in the range of 10-35° C., in the range of 10-30° C., in the range of 10-25° C. or in the range of 10-20° C.

When using bleach systems in traditional detergent compositions, all areas of the textile are not exposed to the bleaching systems to the same extent. Thus, some textile areas may appear less white than areas, where the bleach systems have had free access to act on the textile.

The inventors have surprisingly found that a polypeptide having DNase activity can be used for preparing a textile surface for receiving a bleach system comprising tetraacetylethylenediamine (TAED) or 4-(nonanoyloxy)benzene-1-sulfonate (NOBS). When the polypeptide having DNase activity is used, the textile surface is evenly cleaned and ready for being exposed to the bleach. Thus, the use of a polypeptide having DNase activity together with bleach systems the textile is cleaner. The textile can be exposed to the polypeptide having DNase activity and bleach system during a laundering process. In one embodiment of the invention, the textile can be exposed to the polypeptide having DNase activity in a first laundering process and to the bleach system in a subsequent laundering process, optionally together with a polypeptide having DNase activity.

The invention further concerns a detergent composition comprising a polypeptide having deoxyribonuclease (DNase) activity, a anionic surfactant, and a bleach system comprising tetraacetylethylenediamine (TAED) or 4-(nonanoyloxy)benzene-1-sulfonate (NOBS).

In a preferred embodiment of the invention, the bleach system comprises tetraacetylethylenediamine (TAED) and percarbonate.

The concentration of percarbonate in the wash liquor or in the detergent composition is above five times the concentration of tetraacetylethylenediamine (TAED).

The proportion between tetraacetylethylenediamine (TAED) and percarbonate is important. In one embodiment of the invention, the proportion is above 1:5. In one embodiment, the proportion is in the range of from 1:5.5 to 1:10, from 1:6 to 1:10, from 1:6.1 to 1:10 from 1:6.2 to 1:10, from 1:6.3 to 1:10, from 1:6.3 to 1:8, from 1:6.3 to 1:7 or from 1:6.3 to 1:6.8. In a preferred embodiment, the proportion between tetraacetylethylenediamine (TAED) and percarbonate is 1:6.3.

The detergent composition or the wash liquor further comprises a builder which is not a phosphate builder because phosphate builders have a negative impact on the environment. The builder is selected from sodium carbonate, sodium silicate, zeolite and sodium citrate.

The anionic surfactant used in the laundering method and in the detergent composition is selected from the group consisting of: sulfates and sulfonates, such as linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

In a preferred embodiment, the detergent composition comprises surfactants selected from the group consisting of linear alkylbenzenesulfonates (LAS), alpha-olefinsulfonates (AOS) and alkyl sulfates (AS).

The detergent composition or the wash liquor can further comprise one or more enzymes selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, chlorophyllases, amylases, perhydrolases, peroxidases and xanthanase.

The concentration of the polypeptide having DNase activity in the wash liquor is typically in the range of 0.00004-100 ppm enzyme protein, such as in the range of 0.00008-100 ppm, in the range of 0.0001-100 ppm, in the range of 0.0002-100 ppm, in the range of 0.0004-100 ppm, in the range of 0.0008-100 ppm, in the range of 0.001-100 ppm enzyme protein, 0.01-100 ppm enzyme protein, preferably 0.01-50 ppm enzyme protein, preferably 0.05-50 ppm enzyme protein, more preferably 0.1-50 ppm enzyme protein, more preferably 0.2-50 ppm enzyme protein, more preferably 0.2-30 ppm enzyme protein, more preferably 0.2-10 ppm enzyme protein, more preferably 0.1-30 ppm enzyme protein, more preferably 0.5-20 ppm enzyme protein, and most preferably 0.5-10 ppm enzyme protein.

In a detergent composition, the polypeptide having DNase activity should be present in an amount corresponding to at least 0.002 mg of DNase protein per gram of detergent composition, such as at least 0.004 mg of DNase protein, at least 0.006 mg of DNase protein, at least 0.008 mg of DNase protein, at least 0.01 mg of DNase protein, at least 0.1 mg of protein, at least 0.2 mg of protein, at least 1 mg of protein, at least 10 mg of protein, at least 20 mg of protein, at least 30 mg of protein, at least 40 mg of protein, at least 50 mg of protein, at least 60 mg of protein, at least 70 mg of protein, at least 80 mg of protein, at least 90 mg of protein, at least 100 mg of protein, such as in the range of 80-100 mg of protein per gram detergent composition. Thus, the detergent composition may comprise at least 0.00008% DNase protein, preferably at least 0.002%, 0.003%, 0.004%, 0.005%, 0.006%, 0.008%, 0.01%, 0.02%, 0.03%, 0.05%, 0.1%, 0.2%, 0.3%, 0.4%, 0.6%, 0.7%, 0.8%, 0.9% or 1.0% of DNase protein.

The polypeptide having DNase activity can be of animal, vegetable or microbial origin. In one embodiment the polypeptide is of bacterial or fungal origin.

The polypeptide having DNase activity is selected from the group consisting of: a polypeptide having at least 80% sequence identity to the polypeptide of SEQ ID NO: 1, a polypeptide having at least 80% sequence identity to the polypeptide of SEQ ID NO: 2, a polypeptide having at least 80% sequence identity to the polypeptide of SEQ ID NO: 3, a polypeptide having at least 80% sequence identity to the polypeptide of SEQ ID NO: 4, a polypeptide having at least 80% sequence identity to the polypeptide of SEQ ID NO: 5 and a polypeptide having at least 80% sequence identity to the polypeptide of SEQ ID NO: 6.

In one embodiment of the invention, the polypeptide has at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 1. In one embodiment, one or more amino acids have been substituted, deleted or inserted with the proviso that the deoxyribonuclease activity is maintained, substantially maintained or increased. In a further embodiment, up to 10 amino acids, e.g., 1, 2, 3, 4, 5, 6, 7, 8, 9, or 10, have been substituted, deleted or inserted.

In one embodiment of the invention, the polypeptide has at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 2. In one embodiment, one or more amino acids have been substituted, deleted or inserted with the proviso that the deoxyribonuclease activity is maintained, substantially maintained or increased. In a further embodiment, up to 10 amino acids, e.g., 1, 2, 3, 4, 5, 6, 7, 8, 9, or 10, have been substituted, deleted or inserted.

In one embodiment of the invention, the polypeptide has at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 3. In one embodiment, one or more amino acids have been substituted, deleted or inserted with the proviso that the deoxyribonuclease activity is maintained, substantially maintained or increased. In a further embodiment, up to 10 amino acids, e.g., 1, 2, 3, 4, 5, 6, 7, 8, 9, or 10, have been substituted, deleted or inserted.

In one embodiment of the invention, the polypeptide has at least 85%, at least 90%, at least 91%, at least 92%, at least 93%, at least 94%, at least 95%, at least 96%, at least 97%, at least 98%, at least 99% or 100% sequence identity the polypeptide of SEQ ID NO: 4. In one embodiment, one or more amino acids have been substituted, deleted or inserted with the proviso that the deoxyribonuclease activity is maintained, substantially maintained or increased. In a further embodiment, up to 10 amino acids, e.g., 1, 2, 3, 4, 5, 6, 7, 8, 9, or 10, have been substituted, deleted or inserted.

In one embodiment of the invention, the polypeptide has at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 5. In one embodiment, one or more amino acids have been substituted, deleted or inserted with the proviso that the deoxyribonuclease activity is maintained, substantially maintained or increased. In a further embodiment, up to 10 amino acids, e.g., 1, 2, 3, 4, 5, 6, 7, 8, 9, or 10, have been substituted, deleted or inserted.

In one embodiment of the invention, the polypeptide has at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6. In one embodiment, one or more amino acids have been substituted, deleted or inserted with the proviso that the deoxyribonuclease activity is maintained, substantially maintained or increased. In a further embodiment, up to 10 amino acids, e.g., 1, 2, 3, 4, 5, 6, 7, 8, 9, or 10, have been substituted, deleted or inserted.

In one embodiment of the present invention, the polypeptide having deoxyribonuclease activity is selected from the group consisting of a polypeptide comprising or consisting of the mature polypeptide of SEQ ID NO: 1, a polypeptide comprising or consisting of the amino acid sequence of in SEQ ID NO: 2, a polypeptide comprising or consisting of the amino acid sequence of in SEQ ID NO: 3, a polypeptide comprising or consisting of the mature polypeptide of SEQ ID NO: 4, a polypeptide comprising or consisting of the mature polypeptide of SEQ ID NO: 5 and a polypeptide comprising or consisting of the mature polypeptide of SEQ ID NO: 6. In a further embodiment, the detergent composition comprises a polypeptide consisting of the amino acid sequence of in SEQ ID NO: 2 and a polypeptide consisting of the amino acid sequence of in SEQ ID NO: 3.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for DNase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Detergent Compositions

In one embodiment, the invention is directed to detergent compositions comprising an enzyme of the present invention in combination with one or more additional cleaning composition components. The choice of additional components is within the skill of the artisan and includes conventional ingredients, including the exemplary non-limiting components set forth below.

### Surfactants

The detergent composition comprises one or more surfactants, of which at least one surfactant is anionic. Other surfactants may be anionic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein, the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein, the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein, the detergent will usually contain from about 0% to about 40% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein, the detergent will usually contain from about 0% to about 40% by weight of a zwitterionic surfactant. Non-limiting examples of zwitterionic surfactants include betaines such as alkyldimethylbetaines, sulfobetaines, and combinations thereof.

### Builders and Co-Builders

The detergent composition may contain about 0-65% by weight, such as about 5% to about 50% of a detergent builder or co-builder, or a mixture thereof. The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Zeolites

A preferred class of zeolites is characterized as“intermediate”silicate/aluminate zeolites. The intermediate zeolites are characterized by SiOx/A10z molar ratios of less than about 10. Preferably the molar ratio of SiO₂/A102 ranges from about 2 to about 10. The intermediate zeolites can have an advantage over the “high”zeolites. The intermediate zeolites have a higher affinity for amine-type odors, they are more weight efficient for odor absorption because they have a larger surface area, and they are more moisture tolerant and retain more of their odor absorbing capacity in water than the high zeolites. A wide variety of intermediate zeolites suitable for use herein are commercially available as Valfor® CP301-68, Valfor® 300-63, Valfor® CP300-35, and Valfor®CP300-56, available from PQ Corporation, and the CBV100® series of zeolites from Conteka.

Zeolite materials marketed under the trade name Absents® and Smellrite®, available from The Union Carbide Corporation and UOP are also preferred. Such materials are preferred over the intermediate zeolites for control of sulfur-containing odors, e.g. thiols, mercaptans. When zeolites are used as odor control agents in compositions that are to be sprayed onto surfaces, the zeolite material preferably has a particle size of less than about 10 microns and is present in the composition at a level of less than about 1% by weight of the composition.

### Bleaching Systems

The detergent may contain 0-30% by weight, such as about 1% to about 20%, of a bleaching system. Any bleaching system known in the art for use in detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particulary preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

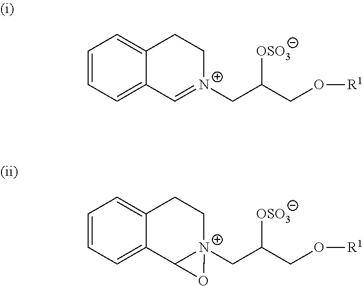

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably, the bleach component comprises a source of peracid in addition to bleach catalyst, particularly organic bleach catalyst. The source of peracid may be selected from (a) preformed peracid; (b) percarbonate, perborate or persulfate salt (hydrogen peroxide source) preferably in combination with a bleach activator; and (c) perhydrolase enzyme and an ester for forming peracid in situ in the presence of water in a textile treatment step.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Enzymes

The detergent additive as well as the detergent composition may comprise one or more additional enzymes such as a protease, lipase, cutinase, an amylase, carbohydrase, cellulase, pectinase, mannanase, arabinase, galactanase, xylanase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases:

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes NS) Carezyme Premium™ (Novozymes NS), Celluclean™ (Novozymes NS), Celluclean Classic™ (Novozymes NS), Cellusoft™ (Novozymes A/S), Whitezyme™ (Novozymes NS), Clazinase™, and Puradax HA™ (Genencor International Inc.), and KAC-500(B)™ (Kao Corporation).

### Proteases:

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those obtained from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases obtained from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those obtained from Bacillus amyloliquefaciens.

Examples of useful proteases are the protease variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the protease variants with alterations in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 corresponding to the positions in BPN′ i.e. BPN′ numbering. More preferred the protease variants are variants of a subtilase variants which comprise one or more of the mutations: S3T, V4I, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering). The protease variants are preferably variants of the Bacillus Lentus protease (Savinase®) shown in SEQ ID NO 1 of WO2016/001449, or of the Bacillus amylolichenifaciens protease (BPN′) shown in SEQ ID NO 2 of WO2016/001449. The protease variants preferably have at least 80% sequence identity to SEQ ID NO 1 or 2 of WO2016/001449. The term “BPN′ numbering” has it common meaning within the protease field and includes the numbering according to the alignment of Savinase and BPN′ as shown in WO1991/000345. The amino acid preceding a position is the amino acid present in sequence of the protease Savinase e.g. shown in SEQ ID NO 1 of WO2016/001449 it is clear to the person skilled in the art that the amino acid to be replaced or deleted can be any amino acid and depends on the parent protease.

The composition of the invention may also preferably comprise a protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737.

Suitable commercially available protease enzymes include those sold under the trade names: Alcalase®, DuralaseTm, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes NS), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes NS), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases:

Suitable amylases which can be used together with the enzyme of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase obtained from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase obtained from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K, wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes NS), and Rapidase™, Purastar/Effectenz™, Powerase and Preferenz S100 (from Genencor International Inc./DuPont).

### Peroxidases/Oxidases:

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment obtained therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In a preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment obtained therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be obtained from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase obtained from Coprinopsis or Myceliophthora is preferred; in particular a laccase obtained from Coprinopsis cinerea, as disclosed in WO 97/08325; or from Myceliophthora thermophila, as disclosed in WO 95/33836.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Other Materials

Any detergent components known in the art for use in detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in detergents may be utilized. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose derivatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The DNase of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, DNase, optionally one or more additional enzymes, a protease inhibitor, and a salt of a monovalent cation and an organic anion may be prepared and the mixture is then plodded. The DNase and optional additional enzymes may be added at the same time as the protease inhibitor for example in liquid form. Besides the mixing step and the plodding step, the process may further comprise the steps of milling, extruding, cutting, stamping, cooling and/or wrapping.

### Formulation of Enzyme in Co-Granule

The DNase may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise a DNase and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

The invention is further summarized in the following paragraphs:
1. A method for laundering a textile comprising the steps of:
   a) Contacting the textile to a wash liquor comprising a polypeptide having DNase activity, an anionic surfactant and a bleach system comprising tetraacetylethylenediamine (TAED) or 4-(nonanoyloxy)benzene-1-sulfonate (NOBS); and optionally an optical brightener; and
   b) Optionally rinsing the textile,

wherein the wash liquor has a temperature in the range of 10-60° C.
2. Method according to paragraph 1, wherein the temperature is in the range of 10-50° C., in the range of 10-45° C., in the range of 10-40° C., in the range of 10-35° C., in the range of 10-30° C., in the range of 10-25° C. or in the range of 10-20° C.
3. Method according to any of paragraphs 1-2, wherein the bleach system comprises tetraacetylethylenediamine (TAED) and percarbonate.
4. Method according to paragraph 3, wherein the concentration of percarbonate in the wash liquor is above five times the concentration of tetraacetylethylenediamine (TAED).
5. Method according to any of paragraphs 3 or 4, wherein the proportion between tetraacetylethylenediamine (TAED) and percarbonate is above 1:5.
6. Method according to paragraph 5, wherein the proportion between tetraacetylethylenediamine (TAED) and percarbonate is from 1:5.5 to 1:10.
7. Method according to any of paragraphs 5 or 6, wherein the proportion between tetraacetylethylenediamine (TAED) and percarbonate is from 1:6 to 1:10, from 1:6.1 to 1:10 from 1:6.2 to 1:10, from 1:6.3 to 1:10, from 1:6.3 to 1:8, from 1:6.3 to 1:7 or from 1:6.3 to 1:6.8.
8. Method according to any of paragraphs 5-7, wherein the proportion between tetraacetylethylenediamine (TAED) and percarbonate is 1:6.3.
9. Method according to any of the preceding paragraphs, wherein the composition comprises an optical brightener
10. Method according to any of the preceding paragraphs, wherein the optical brightener is selected from the group consisting of diaminostilbene-sulfonic acid derivatives, diarylpyrazoline derivatives and bisphenyl-distyryl derivatives.
11. Method according to any of the preceding paragraphs, wherein the wash liquor further comprises a builder which is not a phosphate builder.
12. Method according to any of the preceding paragraphs, wherein the builder is selected from sodium carbonate, sodium silicate, zeolite and sodium citrate.
13. Method according to any of the preceding paragraphs, wherein the wash liquor further comprises one or more enzymes selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, chlorophyllases, amylases, perhydrolases, peroxidases and xanthanase.
14. Method according to any of the preceding paragraphs, wherein the textile is rinsed with water or with water comprising a conditioner.
15. Method according to any of the preceding paragraphs, wherein the polypeptide having DNase activity is of animal, vegetable, microbial origin.
16. Method according to paragraph 15, wherein the polypeptide is of bacterial or fungal origin.
17. Method according to any of paragraphs 13-16, wherein the polypeptide having DNase activity is selected from the group consisting of: a polypeptide having at least 80% sequence identity to the polypeptide of SEQ ID NO: 1, a polypeptide having at least 80% sequence identity to the polypeptide of SEQ ID NO: 2, a polypeptide having at least 80% sequence identity to the polypeptide of SEQ ID NO: 3, a polypeptide having at least 80% sequence identity to the polypeptide of SEQ ID NO: 4, a polypeptide having at least 80% sequence identity to the polypeptide of SEQ ID NO: 5 and a polypeptide having at least 80% sequence identity to the polypeptide of SEQ ID NO: 6.
18. Method according to paragraph 17, wherein the polypeptide has at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the polypeptide of SEQ ID NO: 2, the polypeptide of SEQ ID NO: 3, the polypeptide of SEQ ID NO: 4, the polypeptide of SEQ ID NO: 5 or the polypeptide of SEQ ID NO: 6.
19. Method according to any of the preceding paragraphs, wherein the concentration of the polypeptide in the wash liquor is in the range of 0.00004-100 ppm enzyme protein, such as in the range of 0.00008-100 ppm, in the range of 0.0001-100 ppm, in the range of 0.0002-100 ppm, in the range of 0.0004-100 ppm, in the range of 0.0008-100 ppm, in the range of 0.001-100 ppm enzyme protein, in the range of 0.01-100 ppm enzyme protein, in the range of 0.05-50 ppm enzyme protein, in the range of 0.1-50 ppm enzyme protein, in the range of 0.1-30 ppm enzyme protein, in the range of 0.5-20 ppm enzyme protein or in the range of 0.5-10 ppm enzyme protein, preferably 0.01-50 ppm enzyme protein, preferably 0.05-50 ppm enzyme protein, more preferably 0.1-50 ppm enzyme protein, more preferably 0.2-50 ppm enzyme protein, more preferably 0.2-30 ppm enzyme protein, more preferably 0.2-10 ppm enzyme protein, more preferably 0.1-30 ppm enzyme protein, more preferably 0.5-20 ppm enzyme protein, and more preferably 0.5-10 ppm enzyme protein.
20. Method according to any of the preceding paragraphs, wherein the wash liquor comprises the detergent composition according to paragraph 38-57.
21. Use of a polypeptide having DNase activity for preparing a textile surface for receiving a bleach system comprising tetraacetylethylenediamine (TAED) or 4-(nonanoyloxy)benzene-1-sulfonate (NOBS).
22. Use of a polypeptide having DNase activity for preparing a textile surface for receiving an optical brightener, optionally selected from the group consisting of diaminostilbene-sulfonic acid derivatives, diarylpyrazoline derivatives and bisphenyl-distyryl derivatives.
23. Use according to paragraphs 21 to 22, wherein the textile is exposed to the polypeptide having DNase activity in a laundering process.
24. Use according to paragraph 23, wherein the textile is exposed to the polypeptide having DNase activity in a laundering process and is exposed to the bleach system in a subsequent laundering process.
25. Use according to any of paragraphs 21-24, wherein the temperature of the wash liquor during the laundering process is in the range of 10-60° C.
26. Use according to paragraph 25, wherein the temperature is in the range of 10-50° C., in the range of 10-45° C., in the range of 10-40° C., in the range of 10-35° C., in the range of 10-30° C., in the range of 10-25° C. or in the range of 10-20° C.
27. Use according to any of the preceding use paragraphs, wherein the bleach system comprises tetraacetylethylenediamine (TAED) and percarbonate.
28. Use according to paragraph 27, wherein the amount of percarbonate in the wash liquor is above five times the amount of tetraacetylethylenediamine (TAED).
29. Use according to any of paragraphs 27 or 28, wherein the proportion between tetraacetylethylenediamine (TAED) and percarbonate is above 1:5.
30. Use according to paragraph 29, wherein the proportion between tetraacetylethylenediamine (TAED) and percarbonate is from 1:5.5 to 1:10.
31. Use according to any of paragraphs 29 or 30, wherein the proportion between tetraacetylethylenediamine (TAED) and percarbonate is from 1:6 to 1:10, from 1:6.1 to 1:10 from 1:6.2 to 1:10, from 1:6.3 to 1:10, from 1:6.3 to 1:8, from 1:6.3 to 1:7 or from 1:6.3 to 1:6.8.
32. Use according to any of paragraphs 29-31, wherein the proportion between tetraacetylethylenediamine (TAED) and percarbonate is 1:6.3.
33. Use according to any of paragraphs 21-32, wherein the polypeptide having DNase activity is of animal, vegetable, microbial origin.
34. Use according to paragraph 33, wherein the polypeptide is of bacterial or fungal origin.
35. Use according to paragraph 34, wherein the polypeptide e having DNase activity is selected from the group consisting of a polypeptide having at least 80% sequence identity to the polypeptide of SEQ ID NO: 1, a polypeptide having at least 80% sequence identity to the polypeptide of SEQ ID NO: 2, a polypeptide having at least 80% sequence identity to the polypeptide of SEQ ID NO: 3, a polypeptide having at least 80% sequence identity to the polypeptide of SEQ ID NO: 4, a polypeptide having at least 80% sequence identity to the polypeptide of SEQ ID NO: 5 and a polypeptide having at least 80% sequence identity to the polypeptide of SEQ ID NO: 6.
36. Use according to paragraph 35, wherein the polypeptide has at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the polypeptide of SEQ ID NO: 2, the polypeptide of SEQ ID NO: 3, the polypeptide of SEQ ID NO: 4, the polypeptide of SEQ ID NO: 5 or the polypeptide of SEQ ID NO: 6.
37. Use according to any of paragraphs 21-35, wherein the wash liquor comprises the detergent composition according to paragraphs 38-57.
38. A detergent composition comprising a polypeptide having deoxyribonuclease (DNase) activity, a anionic surfactant, a bleach system comprising tetraacetylethylenediamine (TAED) or 4-(nonanoyloxy)benzene-1-sulfonate (NOBS) and optionally an optical brightener.
39. Composition according to paragraph 38, wherein the bleach system comprises tetraacetylethylenediamine (TAED) and percarbonate.
40. Composition according to paragraph 39, wherein the concentration of percarbonate in the wash liquor is above five times the concentration of tetraacetylethylenediamine (TAED).
41. Composition according to paragraph 40, wherein the proportion between tetraacetylethylenediamine (TAED) and percarbonate is above 1:5.
42. Composition according to any of paragraphs 40-41, wherein the proportion between tetraacetylethylenediamine (TAED) and percarbonate is from 1:5.5 to 1:10.
43. Composition according to any of paragraphs 40-42, wherein the proportion between tetraacetylethylenediamine (TAED) and percarbonate is from 1:6 to 1:10, from 1:6.1 to 1:10 from 1:6.2 to 1:10, from 1:6.3 to 1:10, from 1:6.3 to 1:8, from 1:6.3 to 1:7 or from 1:6.3 to 1:6.8.
44. Composition according to any of paragraphs 40-43, wherein the proportion between tetraacetylethylenediamine (TAED) and percarbonate is 1:6.3.
45. Composition according to any of the preceding composition paragraphs, wherein the anionic surfactant is selected from the group consisting of: sulfates and sulfonates, such as linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.
46. Composition according to paragraph 45, wherein the composition comprises an anionic surfactant selected from the group consisting of linear alkylbenzenesulfonates (LAS), alpha-olefinsulfonates (AOS) and alkyl sulfates (AS).
47. Composition according to any of the preceding paragraphs, wherein the optical brightener is selected from selected from the group consisting of diaminostilbene-sulfonic acid derivatives, diarylpyrazoline derivatives and bisphenyl-distyryl derivatives.
48. Composition according to any of the preceding composition paragraphs, wherein the composition further comprises one or more enzymes selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, chlorophyllases, amylases, perhydrolases, peroxidases and xanthanase.
49. Composition according to any of the preceding composition paragraphs, wherein the polypeptide having DNase activity is of animal, vegetable or microbial origin.
50. Composition according to any of the preceding composition paragraphs, wherein the polypeptide is of bacterial or fungal origin.
51. Composition according to paragraph 50 wherein the polypeptide having DNase activity is selected from the group consisting of a polypeptide having at least 80% sequence identity to the polypeptide of SEQ ID NO: 1, a polypeptide having at least 80% sequence identity to the polypeptide of SEQ ID NO: 2, a polypeptide having at least 80% sequence identity to the polypeptide of SEQ ID NO: 3, a polypeptide having at least 80% sequence identity to the polypeptide of SEQ ID NO: 4, a polypeptide having at least 80% sequence identity to the polypeptide of SEQ ID NO: 5 and a polypeptide having at least 80% sequence identity to the polypeptide of SEQ ID NO: 6.
52. Composition according to paragraph 51, wherein the polypeptide has at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the polypeptide of SEQ ID NO: 2, the polypeptide of SEQ ID NO: 3, the polypeptide of SEQ ID NO: 4, the polypeptide of SEQ ID NO: 5 or the polypeptide of SEQ ID NO: 6.
53. Composition according to any of the preceding composition paragraphs, wherein the composition further comprises a builder which is not a phosphate builder.
54. Composition according to any of the preceding composition paragraphs, wherein the builder is selected from sodium carbonate, sodium silicate, zeolite and sodium citrate.
55. Composition according to any of the preceding composition paragraphs, wherein the composition is a bar, a homogenous tablet, a tablet having two or more layers, a pouch having one or more compartments, a regular or compact powder, a granule, a paste, a gel, or a regular, compact or concentrated liquid.
56. Composition according to any of the preceding composition paragraphs, wherein the composition is a liquid detergent, a powder detergent or a granule detergent.
57. Composition according to any of the preceding composition paragraphs, wherein the composition comprises at least 0.002 mg of DNase protein per gram of detergent composition, at least 0.004 mg of DNase protein, at least 0.006 mg of DNase protein, at least 0.008 mg of DNase protein, at least 0.01 mg of DNase protein, at least 0.1 mg of protein, at least 1 mg of protein, at least 10 mg of protein, at least 20 mg of protein, at least 30 mg of protein, at least 40 mg of protein, at least 50 mg of protein, at least 60 mg of protein, at least 70 mg of protein, at least 80 mg of protein, at least 90 mg of protein or at least 100 mg of protein.
58. Composition according to any of the preceding composition paragraphs, wherein the composition comprises in the range of 80-100 mg of protein per gram detergent composition.

### Assays and Detergent Compositions

### Detergent Compositions

The below mentioned detergent composition can be used in combination with the enzyme of the invention.

### Biotex Black (Liquid)

5-15% Anionic surfactants, <5% Nonionic surfactants, perfume, enzymes, DMDM and hydantoin.

### Composition of Ariel Actilift Colour&Style (Liquid)

Ingredients: 5-15% Anionic surfactants; <5% Non-ionic surfactants, Phosphonates, Soap; Enzymes, Perfumes, Benzisothiazolinone, Methylisothiazolinone, Alpha-isomethyl ionone, Butylphenyl methylpropional, Citronellol, Geraniol, Linalool.

Persil 2 in1 with Comfort Passion Flower Powder

Sodium sulfate, Sodium carbonate, Sodium Dodecylbenzenesulfonate, Bentonite, Sodium Carbonate Peroxide, Sodium Silicate, Zeolite, Aqua, Citric acid, TAED, C12-15 Pareth-7, Stearic Acid, Parfum, Sodium Acrylic Acid/MA Copolymer, Cellulose Gum, Corn Starch Modified, Sodium chloride, Tetrasodium Etidronate, Calcium Sodium EDTMP, Disodium Anilinomorpholinotriazinylaminostilbenesulfonate, Sodium bicarbonate, Phenylpropyl Ethyl Methicone, Butylphenyl Methylpropional, Glyceryl Stearates, Calcium carbonate, Sodium Polyacrylate, Alpha-Isomethyl Ionone, Disodium Distyrylbiphenyl Disulfonate, Cellulose, Protease, Limonene, PEG-75, Titanium dioxide, Dextrin, Sucrose, Sodium Polyaryl Sulphonate, CI 12490, CI 45100, CI 42090, Sodium Thiosulfate, CI 61585.

### Persil Biological Powder

Sucrose, Sorbitol, Aluminum Silicate, Polyoxymethylene Melamine, Sodium Polyaryl Sulphonate, CI 61585, CI 45100, Lipase, Amylase, Xanthan gum, Hydroxypropyl methyl cellulose, CI 12490, Disodium Distyrylbiphenyl Disulfonate, Sodium Thiosulfate, CI 42090, Mannanase, CI 11680, Etidronic Acid, Tetrasodium EDTA.

### Persil Biological Tablets

Sodium carbonate, Sodium Carbonate Peroxide, Sodium bicarbonate, Zeolite, Aqua, Sodium Silicate, Sodium Lauryl Sulfate, Cellulose, TAED, Sodium Dodecylbenzenesulfonate, Hemicellulose, Lignin, Lauryl Glucoside, Sodium Acrylic Acid/MA Copolymer, Bentonite, Sodium chloride, Parfum, Tetrasodium Etidronate, Sodium sulfate, Sodium Polyacrylate, Dimethicone, Disodium Anilinomorpholinotriazinylaminostilbenesulfonate, Dodecylbenzene Sulfonic Acid, Trimethylsiloxysilicate, Calcium carbonate, Cellulose, PEG-75, Titanium dioxide, Dextrin, Protease, Corn Starch Modified, Sucrose, CI 12490, Sodium Polyaryl Sulphonate, Sodium Thiosulfate, Amylase, Kaolin,

### Persil Colour Care Biological Powder

Subtilisin, Imidazolidinone, Hexyl Cinnamal, Sucrose, Sorbitol, Aluminum Silicate, Polyoxymethylene Melamine, CI 61585, CI 45100, Lipase, Amylase, Xanthan gum, Hydroxypropyl methyl cellulose, CI 12490, Disodium Distyrylbiphenyl Disulfonate, Sodium Thiosulfate, CI 42090, Mannanase, CI 11680, Etidronic Acid, Tetrasodium EDTA.

### Persil Colour Care Biological Tablets

Sodium bicarbonate, Sodium carbonate, Zeolite, Aqua, Sodium Silicate, Sodium Lauryl Sulfate, Cellulose Gum, Sodium Dodecylbenzenesulfonate, Lauryl Glucoside, Sodium chloride, Sodium Acrylic Acid/MA Copolymer, Parfum, Sodium Thioglycolate, PVP, Sodium sulfate, Tetrasodium Etidronate, Sodium Polyacrylate, Dimethicone, Bentonite, Dodecylbenzene Sulfonic Acid, Trimethylsiloxysilicate, Calcium carbonate, Cellulose, PEG-75, Titanium dioxide, Dextrin, Protease, Corn Starch Modified, Sucrose, Sodium Thiosulfate, Amylase, CI 74160, Kaolin.

### Persil Dual Action Capsules Bio

MEA-Dodecylbenzenesulfonate, MEA-Hydrogenated Cocoate, C12-15 Pareth-7, Dipropylene Glycol, Aqua, Tetrasodium Etidronate, Polyvinyl Alcohol, Glycerin, Aziridine, homopolymer ethoxylated, Propylene glycol, Parfum, Sodium Diethylenetriamine Pentamethylene Phosphonate, Sorbitol, MEA-Sulfate, Ethanolamine, Subtilisin, Glycol, Butylphenyl Methylpropional, Boronic acid, (4-formylphenyl), Hexyl Cinnamal, Limonene, Linalool, Disodium Distyrylbiphenyl Disulfonate, Alpha-Isomethyl Ionone, Geraniol, Amylase, Polymeric Blue Colourant, Polymeric Yellow Colourant, Talc, Sodium chloride, Benzisothiazolinone, Mannanase, Denatonium Benzoate.

Persil 2 in1 with Comfort Sunshiny Days Powder

Sodium sulfate, Sodium carbonate, Sodium Dodecylbenzenesulfonate, Bentonite, Sodium Carbonate Peroxide, Sodium Silicate, Zeolite, Aqua, Citric acid, TAED, C12-15 Pareth-7, Parfum, Stearic Acid, Sodium Acrylic Acid/MA Copolymer, Cellulose Gum, Corn Starch Modified, Sodium chloride, Tetrasodium Etidronate, Calcium Sodium EDTMP, Disodium Anilinomorpholinotriazinylaminostilbenesulfonate, Sodium bicarbonate, Phenylpropyl Ethyl Methicone, Butylphenyl Methylpropional, Glyceryl Stearates, Calcium carbonate, Sodium Polyacrylate, Geraniol, Disodium Distyrylbiphenyl Disulfonate, Cellulose, Protease, PEG-75, Titanium dioxide, Dextrin, Sucrose, Sodium Polyaryl Sulphonate, CI 12490, CI 45100, CI 42090, Sodium Thiosulfate, CI 61585.

Persil Small & Mighty 2in1 with Comfort Sunshiny Days

Aqua, C12-15 Pareth-7, Sodium Dodecylbenzenesulfonate, Propylene glycol, Sodium Hydrogenated Cocoate, Triethanolamine, Glycerin, TEA-Hydrogenated Cocoate, Parfum, Sodium chloride, Polyquaternium-10, PVP, Polymeric Pink Colourant, Sodium sulfate, Disodium Distyrylbiphenyl Disulfonate, Butylphenyl Methylpropional, Styrene/Acrylates Copolymer, Hexyl Cinnamal, Citronellol, Eugenol, Polyvinyl Alcohol, Sodium acetate, Isopropyl alcohol, Polymeric Yellow Colourant, Sodium Lauryl Sulfate.

### Persil Small & Mighty Bio

Aqua, MEA-Dodecylbenzenesulfonate, Propylene glycol, Sodium Laureth Sulfate, C12-15 Pareth-7, TEA-Hydrogenated Cocoate, MEA-Citrate, Aziridine homopolymer ethoxylated, MEA-Etidronate, Triethanolamine, Parfum, Acrylates Copolymer, Sorbitol, MEA-Sulfate, Sodium Sulfite, Disodium Distyrylbiphenyl Disulfonate, Butylphenyl Methylpropional, Styrene/Acrylates Copolymer, Citronellol, Sodium sulfate, Peptides, salts, sugars from fermentation (process), Subtilisin, Glycerin, Boronic acid, (4-formylphenyl), Geraniol, Pectate Lyase, Amylase, Sodium Lauryl Sulfate, Mannanase, CI 42051.

### Persil Small & Mighty Capsules Biological

MEA-Dodecylbenzenesulfonate, MEA-Hydrogenated Cocoate, C12-15 Pareth-7, Dipropylene Glycol, Aqua, Glycerin, Polyvinyl Alcohol, Parfum, Aziridine homopolymer ethoxylated, Sodium Diethylenetriamine Pentamethylene Phosphonate, Propylene glycol, Sorbitol, MEA-Sulfate, Ethanolamine, Subtilisin, Glycol, Butylphenyl Methylpropional, Hexyl Cinnamal, Starch, Boronic acid, (4-formylphenyl), Limonene, Linalool, Disodium Distyrylbiphenyl Disulfonate, Alpha-Isomethyl lonone, Geraniol, Amylase, Talc, Polymeric Blue Colourant, Sodium chloride, Benzisothiazolinone, Denatonium Benzoate, Polymeric Yellow Colourant, Mannanase.

### Persil Small & Mighty Capsules Colour Care

MEA-Dodecylbenzenesulfonate, MEA-Hydrogenated Cocoate, C12-15 Pareth-7, Dipropylene Glycol, Aqua, Glycerin, Polyvinyl Alcohol, Parfum, Aziridine homopolymer ethoxylated, Sodium Diethylenetriamine Pentamethylene Phosphonate, Propylene glycol, MEA-Sulfate, Ethanolamine, PVP, Sorbitol, Butylphenyl Methylpropional, Subtilisin, Hexyl Cinnamal, Starch, Limonene, Linalool, Boronic acid, (4-formylphenyl), Alpha-Isomethyl lonone, Geraniol, Talc, Polymeric Blue Colourant, Denatonium Benzoate, Polymeric Yellow Colourant.

### Persil Small & Mighty Colour Care

Aqua, MEA-Dodecylbenzenesulfonate, Propylene glycol, Sodium Laureth Sulfate, C12-15 Pareth-7, TEA-Hydrogenated Cocoate, MEA-Citrate, Aziridine homopolymer ethoxylated, MEA-Etidronate, Triethanolamine, Parfum, Acrylates Copolymer, Sorbitol, MEA-Sulfate, Sodium Sulfite, Glycerin, Butylphenyl Methylpropional, Citronellol, Sodium sulfate, Peptides, salts, sugars from fermentation (process), Styrene/Acrylates Copolymer, Subtilisin, Boronic acid, (4-formylphenyl), Geraniol, Pectate Lyase, Amylase, Sodium Lauryl Sulfate, Mannanase, CI 61585, CI 45100.

### Composition of Ariel Actilift (Powder)

Ingredients: 5-15% Anionic surfactants, Oxygen-based bleaching agents, <5% Non-ionic surfactants, Phosphonates, Polycarboxylates, Zeolites, Optical brightners, Enzymes, Perfumes, Butylphenyl Methylpropional, Coumarin, Hexyl Cinnamal.

### Composition of Model Detergent T (Powder)

Ingredients: 11% LAS, 2% AS/AEOS, 2% soap, 3% AEO, 15.15% sodium carbonate, 3% sodium slilcate, 18.75% zeolite, 0.15% chelant, 2% sodium citrate, 1.65% AA/MA copolymer, 2.5% CMC and 0.5% SRP (all percentages are w/w).

### Wash Assays

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM or is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines.

In miniLOM, washes are performed in 50 ml test tubes placed in Stuart rotator.

### Enzyme Assays

### Assay I

### Testing of DNase Activity

DNase activity was determined on DNase Test Agar with Methyl Green (BD, Franklin Lakes, N.J., USA), which was prepared according to the manual from supplier. Briefly, 21 g of agar was dissolved in 500 ml water and then autoclaved for 15 min at 121° C. Autoclaved agar was temperated to 48° C. in water bath, and 20 ml of agar was poured into petridishes with and allowed to solidify by incubation o/n at room temperature. On solidified agar plates, 5 μl of enzyme solutions are added, and DNase activity are observed as colorless zones around the spotted enzyme solutions.

### EXAMPLES

### Example 1

Isolating Laundry Specific Bacterial Strains

One strain of Brevundimonas sp. isolated from laundry was used in the present example.

The Brevundimonas sp. was isolated during a study, where the bacterial diversity in laundry after washing at 15, 40 and 60° C., respectively, was investigated. The study was conducted on laundry collected from Danish households. For each wash, 20 g of laundry items (tea towel, towel, dish cloth, bib, T-shirt armpit, T-shirt collar, socks) in the range 4:3:2:2:1:1:1 was used. Washing was performed in a Laundr-O-Meter (LOM) at 15, 40 or 60° C. For washing at 15 and 40° C., Ariel Sensitive White & Color was used, whereas WFK IEC-A* model detergent was used for washing at 60° C. Ariel Sensitive White & Color was prepared by weighing out 5.1 g and adding tap water up to 1000 ml followed by stirring for 5 minutes. WFK IEC-A* model detergent (which is available from WFK Testgewebe GmbH) was prepared by weighing out 5 g and adding tap water up to 1300 ml followed by stirring for 15 min. Washing was performed for 1 hour at 15, 40 and 60° C., respectively, followed by 2 times rinsing with tap water for 20 min at 15° C.

Laundry was sampled immediately after washing at 15, 40 and 60° C., respectively. Twenty grams of laundry was added 0.9% (w/v) NaCl (1.06404; Merck, Damstadt, Germany) with 0.5% (w/w) tween 80 to yield a 1:10 dilution in stomacher bag. The mixture was homogenized using a Stomacher for 2 minutes at medium speed. After homogenization, ten-fold dilutions were prepared in 0.9% (w/v) NaCl. Bacteria were enumerated on Tryptone Soya Agar (TSA) (CM0129, Oxoid, Basingstoke, Hampshire, UK) incubated aerobically at 30° C. for 5-7 days. To suppress growth of yeast and moulds, 0.2% sorbic acid (359769, Sigma) and 0.1% cycloheximide (18079; Sigma) were added. Bacterial colonies were selected from countable plates and purified by restreaking twice on TSA. For long time storage, purified isolates were stored at −80° C. in TSB containing 20% (w/v) glycerol (49779; Sigma).

Preparation of Donor Swatches with Biofilm

Brevundimonas sp. was pre-grown on Tryptone Soya Agar (TSA) (pH 7.3) (CM0131; Oxoid Ltd, Basingstoke, UK) for 2-5 days at 30° C. From a single colony, a loop-full was transferred to 10 mL of TSB and incubated for 1 day at 30° C. with shaking (240 rpm). After propagation, Brevundimonas sp. was pelleted by centrifugation (Sigma Laboratory Centrifuge 6K15) (3000 g at 21° C. in 7 min) and resuspended in 10 mL of TSB diluted twice with water. Optical density (OD) at 600 nm was measured using a spectophometer (POLARstar Omega (BMG Labtech, Ortenberg, Germany). Fresh TSB diluted twice with water was inoculated to an OD₆₀₀ₙₘ of 0.03, and 1.6 mL was added into each well of a 12-well polystyrene flat-bottom microplate (3512; Corning Incorporated, Corning, N.Y., USA), in which a round swatch (diameter 2 cm) of sterile Polyester WFK30A was placed. After incubation (24 h at 15° C. with shaking (100 rpm), swatches were rinsed twice with 0.9% (w/v) NaCl.

### Example 2

### Wash Performance of Polypeptide Having DNAse Activity in the Presence of Bleach System

### Materials and Methods

### Preparation of Biofilm Swatches

Round swatches (diameter 2 cm) of sterile Polyester WFK30A with Brevundimonas sp. biofilm was prepared as described in Example 1.

### Washing Experiment

Wash liquor of Powder Model detergent T without bleach was prepared by dissolving detergent (5.33 g/l) in water with hardness 15° dH. The AEO Biosoft N25-7 (NI) (0.16 g/l) component of model detergent T was added separately. Pigment soil (Pigmentschmutz, 09V, wfk, Krefeld, Germany) (0.7 g/L) was added to the wash liquor. Various amounts of the bleach system consisting of TAED and percarbonate were weighed out and dissolved in the wash liquor by stirring on a magnetic stirrer for 5 min. 10 ml of wash liquor was added to a 50 ml tube in which five rinsed swatches with Brevundimonas sp. and five sterile polyester (WFK30A) swatches. In washes where A. oryzae DNase was included, DNase (0.5 ppm) was added to the wash liquor. Washing was performed in Stuart rotator for 1 hour at 30° C. (MiniLOM assay as described herein). After washing, swatches with Brevundimonas sp. were rinsed twice in tap water and dried on filter paper over night. Color difference (L values) was measured using a Color Eye (Macbeth Color Eye 7000 reflectance spectrophotometer). The measurements were made without UV in the incident light and the L value from the CIE Lab color space was extracted.

TABLE 1
Wash performance of DNAse in the presence of a bleach system.

| | TAED | Percar- | | | |
|---|---|---|---|---|---|
| Model | (g/100 g | bonate | | | Delta L value |
| detergent | deter- | (g/100 g | DNAse | L | (L value − L |
| T (g/l) | gent) | detergent) | (ppm) | value | value_{(no bleach, no DNase}) |
| 5.49 | 0 | 0 | 0 | 83.8 | |
| 5.49 | 0 | 0 | 0.5 | 88.7 | 5.3 |
| 5.49 | 1.5 | 9.45 | 0 | 84.1 | 0.7 |
| 5.49 | 1.5 | 9.45 | 0.5 | 90.1 | 6.8 |
| 5.49 | 3.0 | 18.9 | 0 | 84.3 | 0.9 |
| 5.49 | 3.0 | 18.9 | 0.5 | 90.6 | 7.3 |
| 5.49 | 3.75 | 23.625 | 0 | 85.5 | 2.1 |
| 5.49 | 3.75 | 23.625 | 0.5 | 89.9 | 6.5 |
| 5.49 | 4.5 | 28.35 | 0 | 84.8 | 1.4 |
| 5.49 | 4.5 | 28.35 | 0.5 | 90.9 | 7.5 |
| 5.49 | 6.0 | 37.8 | 0 | 85.7 | 2.3 |
| 5.49 | 6.0 | 37.8 | 0.5 | 90.3 | 6.9 |
| 5.49 | 9.0 | 56.7 | 0 | 86.2 | 2.9 |
| 5.49 | 9.0 | 56.7 | 0.5 | 91.3 | 7.9 |

The data presented in Table 1 demonstrate that the wash performance of the A. oryzae DNase of the present invention is not affected by the presence of a bleaching system. Further, the data demonstrate that the A. oryzae DNase contribution to the whiteness of the textile exceeds that of the bleaching system.

### Example 3 Wash Performance of Polypeptide Having DNAse Activity in the Presence of Bleach System

The effect of DNAse and Bleach and e.g. synergistic effects may be shown in a setup where textile is soiled with a combination of DNA and typical bleachable soil blueberry juice. The textile is preferred to be Poly Cotton 50/50 Jersey knittedT-7422 but could also be textile of either WFK20A cotton polyester or wfk 10A cotton or wfk 30A polyester.

### Preparation of DNA/Blue Berry Swatches:

Round swatches (diameter 2 cm) of sterile Polyester WFK30A with Salmon DNA or blueberry juice, DNA with added blueberry juice or DNA mixed with blueberry juice before addition to textile may be prepared.

DNA swatches: swatches 1.5 g Deoxyribonucleic acid sodium from Salmon testes; DNA (Sigma 1626) is weighed out and added to 100 ml milliQ water in a bluecap bottle. Put in a magnetic stirrer. Stir to 60 minutes on max speed until it is homogeneous. Textile is smothered into the solution and soaked for 2 min and rolled dry in a rubber roller. It is dried at 35° C. for 5 hours and left at room temperature overnight. Punch out the swatch to 2 cm circular swatches.

Blueberry juice swatches maybe prepared as described: 11 ml blueberry juice is added to 100 ml100 ml milliQ water in a bluecap bottle. Put in a magnetic stirrer. Stir to 60 minutes on max speed until it is homogeneous. Textile is smothered into the solution and soaked for 2 min and rolled dry in a rubber roller. It is dried at 35° C. for 5 hours and left at room temperature overnight. Punch out the swatch to 2 cm circular swatches

DNA+ blueberry juice swatches 1:1.5 g Deoxyribonucleic acid sodium from Salmon testes; DNA (Sigma 1626) is weighed out and added to 100 ml milliQ water in a bluecap bottle. Put in a magnetic stirrer. Stir to 60 minutes on max speed until it is homogeneous. Add 11 ml blueberry juice and mix well. Textile is smothered into the solution and soaked for 2 min and rolled dry in a rubber roller. It is dried at 35° C. for 5 hours and left at room temperature overnight. Punch out the swatch to 2 cm circular swatches.

DNA+ blueberry juice swatches 2:1.5 g Deoxyribonucleic acid sodium from Salmon testes; DNA (Sigma 1626) is weighed out and added to 11 ml blueberry juice in a bluecap bottle. Put in a magnetic stirrer. Stir to 60 minutes on max speed until it is homogeneous heat if necessary add 100 ml milliQ water and stir until it is homogeneous. Textile is smothered into the solution and soaked for 2 min and rolled dry in a rubber roller. It is dried at 35° C. for 5 hours and left at room temperature overnight. Punch out the swatch to 2 cm circular swatches

Wash liquor of Powder Model detergent T without bleach may be prepared by dissolving detergent (5.33 g/l) in water with hardness 15° dH. The AEO Biosoft N25-7 (NI) (0.16 g/l) component of model detergent T is added separately. Pigment soil (Pigmentschmutz, 09V, wfk, Krefeld, Germany) (0.7 g/L) is added to the wash liquor. Various amounts of the bleach system consisting of TAED and percarbonate are weighed out and dissolved in the wash liquor by stirring on a magnetic stirrer for 5 min. 10 ml of wash liquor is added to a 50 ml tube in which five rinsed swatches with either DNA swatches, blueberry swatches, DNA/blueberry 1 swatches or blueberry 2 swatches.

Washing with DNA stains +/− pigment soil or Carbon Black will demonstrate the gluing effect of DNA and that Bleach cannot remove the color appearance from the particulates.

Washing with blueberry swatches +/− particulate soil and +/− DNase will show the removal/decrease of color compared to wash without bleach and when particulate soil is present in the wash the effect from adding DNase alone as well as together with bleach.

Washing with DNA blueberry stain 1 will show if bleach will remove the blue berry juice color in a wash without particulate soil present. When the soil is present the soil may adhere and discolor the fabric. When DNase is present alone without particulate soil and without bleach there is no bleaching of the blueberry juice color. When particulate soil is present in a solution with both DNase and bleach the deposition will be avoided and the swatched will be as white as the original swatch is after a wash in clean detergent. When the combination of DNase and Bleach is present in a wash with particulate soil both removal of the coloration from juice/curry as well inhibition of the deposition of particulate soil will be shown because the sticky DNA has been removed, too.

Washing with DNA blueberry stain 2 will show that bleach can destroy the color when there is no particulate present but there will be deposition of particles when it is present. DNase will be able to remove some parts of the DNA with color when there is no soil present. Not total removal. But the combination of DNase and Bleach will be as white as the unsoiled swatch after a wash in clean detergent because the blueberry juice is bleached away and there is no sticky DNA left on the textile to adhere the particulates.

TABLE 2

| | | | |
|---|---|---|---|
| DNA swatch | Without carbon black or | Bleach | No DNase |
| | WFK 09V standard | No Bleach | No DNase |
| | pigment soil | Bleach | 2 ppm DNase |
| | | No Bleach | 2 ppm DNase |
| | Without carbon black or | Bleach | No DNase |
| | WFK 09V standard | No Bleach | No DNase |
| | pigment soil | Bleach | 2 ppm DNase |
| | | No Bleach | 2 ppm DNase |
| Blueberry juice | Without carbon black or | Bleach | No DNase |
| no DNA | WFK 09V standard | No Bleach | No DNase |
| | pigment soil | Bleach | 2 ppm DNase |
| | | No Bleach | 2 ppm DNase |
| | Without carbon black or | Bleach | No DNase |
| | WFK 09V standard | No Bleach | No DNase |
| | pigment soil | Bleach | 2 ppm DNase |
| | | No Bleach | 2 ppm DNase |
| DNA swatch with | Without carbon black or | Bleach | No DNase |
| added Blueberry | WFK 09V standard | No Bleach | No DNase |
| juice | pigment soil | Bleach | 2 ppm DNase |
| | | No Bleach | 2 ppm DNase |
| | Without carbon black or | Bleach | No DNase |
| | WFK 09V standard | No Bleach | No DNase |
| | pigment soil | Bleach | 2 ppm DNase |
| | | No Bleach | 2 ppm DNase |
| DNA treated with | Without carbon black or | Bleach | No DNase |
| Blueberry juice or | WFK 09V standard | No Bleach | No DNase |
| curry swatch and | pigment soil | Bleach | 2 ppm DNase |
| added to swatch | | No Bleach | 2 ppm DNase |
| | Without carbon black or | Bleach | No DNase |
| | WFK 09V standard | No Bleach | No DNase |
| | pigment soil | Bleach | 2 ppm DNase |
| | | No Bleach | 2 ppm DNase |

1. A method for laundering a textile comprising the steps of:
a) Contacting the textile to a wash liquor comprising a polypeptide having DNase activity, an anionic surfactant, a bleach system comprising tetraacetylethylenediamine (TAED) or 4-(nonanoyloxy)benzene-1-sulfonate (NOBS) and optionally an optical brightener; and
b) Optionally rinsing the textile,
wherein the wash liquor has a temperature in the range of 10-60° C.

2. Method according to claim 1, wherein the temperature is in the range of 10-20° C.

3. Method according to claim 1, wherein the bleach system comprises tetraacetylethylenediamine (TAED) and percarbonate.

4. Method according to claim 3, wherein a concentration of percarbonate in the wash liquor is above five times a concentration of tetraacetylethylenediamine (TAED).

5. Method according to claim 1, wherein the wash liquor further comprises a builder which is not a phosphate builder.

6. Method according to claim 1, wherein the method comprises the optical brightener selected from the group consisting of diaminostilbene-sulfonic acid derivatives, diarylpyrazoline derivatives and bisphenyl-distyryl derivatives.

7. Method according to claim 1, wherein the polypeptide having DNase activity is selected from the group consisting of: a polypeptide having at least 80% sequence identity to the polypeptide of SEQ ID NO: 1, a polypeptide having at least 80% sequence identity to the polypeptide of SEQ ID NO: 2, a polypeptide having at least 80% sequence identity to the polypeptide of SEQ ID NO: 3, a polypeptide having at least 80% sequence identity to the polypeptide of SEQ ID NO: 4, a polypeptide having at least 80% sequence identity to the polypeptide of SEQ ID NO: 5 and a polypeptide having at least 80% sequence identity to the polypeptide of SEQ ID NO: 6.

8. Method according to claim 1, wherein a concentration of the polypeptide in the wash liquor is in the range of 0.00004-100 ppm enzyme protein.

9. (canceled)

10. (canceled)

11. (canceled)

12. A detergent composition comprising a polypeptide having deoxyribonuclease (DNase) activity, a anionic surfactant, a bleach system comprising tetraacetylethylenediamine (TAED) or 4-(nonanoyloxy)benzene-1-sulfonate (NOBS) and optionally an optical brightener.

13. Composition according to claim 12, wherein the bleach system comprises tetraacetylethylenediamine (TAED) and percarbonate.

14. Composition according to claim 13, wherein a concentration of percarbonate in the wash liquor is above five times a concentration of tetraacetylethylenediamine (TAED).

15. Composition according to claim 12 comprising the optical brightener, selected from the group consisting of diaminostilbene-sulfonic acid derivatives, diarylpyrazoline derivatives and bisphenyl-distyryl derivatives.

16. Composition according to claim 12, wherein the polypeptide having DNase activity is selected from the group consisting of a polypeptide having at least 80% sequence identity to the polypeptide of SEQ ID NO: 1, a polypeptide having at least 80% sequence identity to the polypeptide of SEQ ID NO: 2, a polypeptide having at least 80% sequence identity to the polypeptide of SEQ ID NO: 3, a polypeptide having at least 80% sequence identity to the polypeptide of SEQ ID NO: 4, a polypeptide having at least 80% sequence identity to the polypeptide of SEQ ID NO: 5 and a polypeptide having at least 80% sequence identity to the polypeptide of SEQ ID NO: 6.

17. Composition according to claim 12, wherein the composition further comprises a builder which is not a phosphate builder.
